(19) United States
(12) Patent Application Publication
(10) Pub. No.: US 2024/0407750 A1
(43) Pub. Date: Dec. 12, 2024

(54) IMPLANTABLE APNOEA DETECTION DEVICE

(71) Applicant: BIOTRONIK SE & Co. KG, Berlin (DE)

(72) Inventors: Christian MOSS, Falkensee (DE); Olaf SKERL, Bad Doberan (DE)

(73) Assignee: BIOTRONIK SE & Co. KG, Berlin (DE)

(21) Appl. No.: 18/717,804

(22) PCT Filed: Dec. 20, 2022
(86) PCT No.: PCT/EP2022/086945

§ 371 Date: Jun. 7, 2024

(30) Foreign Application Priority Data
Dec. 22, 2021 (EP) 21216809.0

(51) Int. Cl. A61B 8/04; A61B 8/08; A61B 8/12

(52) U.S. Cl. CPC A61B 8/04; A61B 8/12; A61B 8/5223

(57) ABSTRACT

An implantable apnoea detection device and an implantable blood pressure monitoring device using such apnoea detection device as well as an apnoea detection arrangement and a blood pressure monitoring arrangement using such devices are presented. The apnoea detection device comprises an ultrasonic transmitter, an ultrasonic receiver and a controller. The apnoea detection device is configured for being implanted into a body of a patient. The ultrasonic transmitter is configured for transmitting ultrasonic signals. The ultrasonic receiver is configured for receiving ultrasonic signals. The controller is configured for generating a phase signal indicating a phase difference between the transmitted ultrasonic signals and the received ultrasonic signals. In the apnoea detection arrangements or the blood pressure monitoring arrangement, the phase signal may be used for determining an occurrence of an apnoea. Eventually, blood pressure signals may be interpreted taking into account the determined occurrence of the apnoea.

### CROSS-REFERENCE TO RELATED APPLICATIONS

This application is the United States National Phase under 35 U.S.C. § 371 of PCT International Patent Application No. PCT/EP2022/086945, filed on Dec. 20, 2022, which claims the benefit of European Patent Application No. 21216809.0, filed on Dec. 22, 2021, the disclosures of which are hereby incorporated by reference herein in their entireties.

### TECHNICAL FIELD

The present invention relates to detecting or monitoring a medical condition of a patient. Particularly, the present invention relates to an implantable blood pressure monitoring device, a blood pressure monitoring arrangement and a method for monitoring a blood pressure of a patient in combination with an implantable apnoea detection device, an apnoea detection arrangement and a method for detecting an apnoea status of a patient.

### BACKGROUND

A medical condition of a patient may be influenced by various physiological parameters. Particularly for patients suffering from medical deficiencies, it may be important to detect any abnormal medical condition at an early stage, in order to enable initiating suitable countermeasures.

For example, the blood pressure within vessels and/or chambers of a patient is usually subject to pressure changes. However, excessive pressure changes or pressure changes occurring during specific situations may be critical for the patient. Accordingly, for patients suffering, for example, from specific deficiencies, the blood pressure may have to be continuously monitored in order to supervise the patient for an occurrence of critical medical conditions. In case of the blood pressure exceeding predefined limits in specific situations, suitable countermeasures may then be initiated accordingly, for example, by suitably supporting the patient's heart and/or blood circuit. For example, if the current blood pressure exceeds over a predefined upper limit, blood pressure reducing measures may be initiated, whereas, if the current blood pressure exceeds under a predefined lower limit, blood pressure increasing measures may be initiated. The upper and lower limits may be set depending on the specific situations, i.e., taking into account whether the patient is currently at rest, at physical or emotional stress, healthy or not, etc.

Approaches for continuously monitoring the blood pressure of a patient have been developed. For example, German Publication No. 10 2015 116 648 A1 discloses an implantable pressure sensor arrangement which may be implanted into an animal or human body.

However, it has been observed that conventionally monitoring a patient's medical condition, for example, by monitoring the blood pressure of the patient may not always result in correctly determining a current medical condition of the patient.

The present disclosure is directed toward overcoming one or more of the above-mentioned problems, though not necessarily limited to embodiments that do.

### SUMMARY

It is an object of the present invention to provide devices, arrangements and methods for monitoring a patient's medical condition with a high degree of reliability. Particularly, it may be an object of the present invention to provide devices, arrangements and methods which allow detecting an apnoea and a blood pressure with a high degree of reliability.

At least such objects may be met with the subject-matter of the independent claims. Advantageous embodiments are defined in the dependent claims as well as the corresponding specification and figures.

According to a first aspect of the present invention, an implantable apnoea detection device is proposed, the device comprising an ultrasonic transmitter, an ultrasonic receiver and a controller. The apnoea detection device is configured for being a part of an implantable pressure sensor device configured for being implanted into a body of a patient. The ultrasonic transmitter is configured for transmitting ultrasonic signals. The ultrasonic receiver is configured for receiving ultrasonic signals. The controller is configured for generating a phase signal indicating a phase difference between the transmitted ultrasonic signals and the received ultrasonic signals.

According to a second aspect of the present invention, an apnoea detection arrangement is proposed, the arrangement comprising an implantable apnoea detection device according to an embodiment of the first aspect of the present invention and an apnoea monitoring device. The apnoea monitoring device is configured to detect an occurrence of an apnoea based on analysing a time-dependency of the phase signal generated by the controller of the apnoea detection device.

According to a third aspect of the present invention, an implantable blood pressure monitoring device is proposed, the device comprising a blood pressure detector and an implantable apnoea detection device according to an embodiment of the first aspect of the present invention. The blood pressure monitoring device is configured for being implanted into a body of a patient. The blood pressure detector is configured for generating a blood pressure signal indicating a blood pressure within a blood vessel and/or a blood chamber in the body of the patient.

According to a fourth aspect of the present invention, a blood pressure monitoring arrangement is proposed, the arrangement comprising an implantable blood pressure monitoring device according to an embodiment of the third aspect of the present invention and a blood pressure observation device. The blood pressure observation device is configured to monitor the blood pressure within the blood vessel and/or the blood chamber of the patient based on the blood pressure signal generated by the blood pressure detector and taking into account the phase signal generated by the controller of the apnoea detection device.

According to a fifth aspect of the present invention, a method for detecting an apnoea status of a patient is proposed, the method comprising at least the following steps, preferably in the indicated order:
transmitting ultrasonic signals by an ultrasonic transmitter implanted within the patient, receiving ultrasonic signals by an ultrasonic receiver implanted within the patient, and detecting an occurrence of the apnoea based on analysing a time-dependency of a phase signal indicating a phase difference between the transmitted ultrasonic signals and the received ultrasonic signals.

According to a sixth aspect of the present invention, a method for monitoring a blood pressure of a patient is proposed, the method comprising at least the following steps, preferably in the indicated order:
repeatedly detecting the blood pressure within one of a blood vessel and a blood chamber of the patient by a blood pressure detector implanted within the patient, detecting an apnoea status of the patient in accordance with an embodiment of the fifth aspect of the present invention,
monitoring the blood pressure based on the detected blood pressure and taking into account the detected apnoea status of the patient.

Ideas underlying embodiments of the present invention may be interpreted as being based, inter alia, on the following observations and recognitions.

Briefly summarised in a non-limiting manner, embodiments of the present invention relate to detecting or monitoring a medical condition of the patient which may be influenced by an occurrence of an apnoea. Particularly, it has been found that the occurrence of an apnoea may significantly influence the blood pressure of the patient. Accordingly, in order to enable distinguishing blood pressure variations resulting from the influence of an apnoea from other blood pressure variations which, for example, could indicate a critical medical condition of the patient requiring initiating countermeasures, it is suggested to continuously or repeatedly detect an apnoea status of the patient. A detected occurrence of an apnoea may then be taken into account, for example, upon monitoring the blood pressure of the patient. The occurrence of the apnoea may be detected by transmitting and receiving ultrasonic signals using an ultrasonic transmitter and an ultrasonic receiver implanted within the patient. The transmitted ultrasonic signals are significantly reflected at a surface of the patient's body and may then be received by the ultrasonic receiver. Therein, a phase difference between the transmitted ultrasonic signals and the received ultrasonic signals generally depends on a distance between the ultrasonic transmitter and receiver, on the one side, and the surface of the patient's body, on the other side. As the surface particularly in a thorax region of the patient's body and/or at an interface within the patient's lung is periodically moving due to respiratory motions, this phase difference is generally changing with a periodicity corresponding to the periodicity of the respiratory motion. However, upon occurrence of an apnoea, the respiratory motion is temporarily interrupted and, accordingly, the phase difference between the transmitted ultrasonic signals and the received ultrasonic signals no more follows the typical periodicity during normal respiration but becomes quasi-stationary. Accordingly, the occurrence of the apnoea may be detected by analysing a time dependency of the phase signal indicating the phase difference between the transmitted and received ultrasonic signals.

In the following, characteristics of embodiments of the present invention will be described in more detail. Therein, a focus will be set on embodiments of the blood pressure monitoring device, arrangement and method according to the third, fourth and sixth aspect of the present invention. Such embodiments advantageously use an information about a detected occurrence of an apnoea as it may be provided with the apnoea detection device, arrangement and method according to the first, second and fifth aspect of the present invention. However, it shall be noted that detecting the occurrence of an apnoea may also be beneficially applied in detecting or monitoring other medical conditions of the patient.

An apnoea is a temporary interruption of a respiration of a patient. For example, an adult human at rest typically breathes between 10 and 20 times per minute, i.e., a respiration cycle generally takes between 3 and 6 seconds. During an apnoea, the patient temporarily stops breathing. Accordingly, a respiration motion such as a periodic motion of the thorax and/or lung of the patient is temporarily interrupted.

The apnoea detection device according to the first aspect of the present invention and the blood pressure monitoring device according to the third aspect of the present invention shall be configured such that they may be implanted into the body of the patient. Particularly, such devices shall be implanted into a vessel or a chamber within the patient. For example, the device may be implanted into a pulmonary artery. Alternatively, the device may be implanted into another vessel close to the heart or directly into a chamber of the heart such as a ventricle or an atrium.

For such implantation purposes, the device may have to be specifically configured with regard to its sizes, materials, shape, functionalities, etc. For example, the dimensions of the device should be sufficiently small such that the device may be accommodated within the intended vessel or chamber. Particularly, a device to be implanted into a vessel may have an elongate shape with a length being, for example, smaller than 50 mm, preferably smaller than 25 mm, and cross-sectional dimensions being smaller than 10 mm, preferably smaller than 5 mm. Materials used in the implantable device may have to be selected such as to comply with environmental conditions in the implanted state. For example, materials should be non-toxic, non-corrosive, chemically stable and/or sufficiently mechanically rigid and loadable. This may be particularly relevant with regard to materials used for a housing of the implantable device, such materials being in direct contact with living tissue and/or body liquids. Such housing may accommodate other components of the implantable device in a tight and preferably hermetically sealed manner. The shape of such housing may be adapted to available spatial conditions at an implantation site. Particularly, the shape may be rounded, i.e., without sharp edges, in order to reduce any risk of injuries. Furthermore, the implantable device may be adapted for operating autonomously. Particularly, the implantable device may have its own energy supply. For example, the implantable device may include batteries and/or capacitors. Furthermore, the implantable device may comprise data or signal communication components for communicating with other devices, particularly for communicating with devices external to the patient's body.

The implantable apnoea detection device comprises an ultrasonic transmitter and an ultrasonic receiver. The ultrasonic transmitter and the ultrasonic receiver may be provided as separate components. Alternatively, the ultrasonic transmitter and the ultrasonic receiver may be integrated into a single component such as an ultrasonic transceiver. The ultrasonic transmitter is configured for emitting ultrasonic signals, i.e., acoustic signals having a frequency above an audible frequency range of humans. The ultrasonic transmitter may be configured to transduce electrical signals in an ultrasonic frequency range into acoustic signals, particularly into pressure waves, in the same ultrasonic frequency range. The electrical signals may be provided by a controller. The ultrasonic receiver is configured for receiving ultrasonic acoustic signals and transduce these signals into electric signals. The resulting electric signals may be transmitted to the controller. The electric signals may indicate both an amplitude and a phase of the ultrasonic signals. Accordingly, the controller may determine the phase of the emitted ultrasonic signal and the phase of the received ultrasonic signal and may generate a phase signal indicating a phase difference between these two signals.

Generally, the ultrasonic transmitter and the ultrasonic receiver may be any components which, due to their physical characteristics, enable emitting and receiving ultrasonic acoustic signals. For example, the ultrasonic transmitter and receiver may be micro-electromechanical systems (MEMS).

According to a preferred embodiment, the ultrasonic transmitter and/or the ultrasonic receiver comprise or are piezoelectric micromachined ultrasonic transducers (PMUT). Compared to conventional bulk thickness-mode piezoelectric transducers, such PMUTs may provide several advantages such as mall device size and/or low cost due to their MEMS-based fabrication. Furthermore, PMUTs may enable transmitting and receiving ultrasonic signals within a large frequency range. Particularly, although being small in size, a PMUT may emit signals at relatively low ultrasonic frequencies of, for example, less than 1 Mhz or even less than 100 KHz.

The controller of the apnoea detection device is specifically configured for generating a phase signal indicating the phase difference between the transmitted ultrasonic signals and the received ultrasonic signals. In other words, the controller may determine the phase difference between a phase of the ultrasonic signals transmitted by the ultrasonic transmitter and a phase of the ultrasonic signals received by the ultrasonic receiver. For such purpose, the transmitted signals and the received signals may, for example, be superimposed. A signal resulting from such superimposition may represent the phase difference between both signals.

Generally, ultrasonic signals emitted by the ultrasonic transmitter propagate throughout tissue within the patient's body. Therein, a propagation velocity, i.e., sound velocity, typically is between 1000 m/s and 2000 m/s, depending on, e.g., physical tissue characteristics, and may be assumed to be generally approximately 1500 m/s.

Generally, the ultrasonic signals are attenuated during their propagation. An attenuation of the ultrasonic signals generally depends on a frequency of these signals. Typically, the attenuation is smaller for smaller frequencies. For example, an attenuation of less than 10 dB, preferably less than 5 dB, may apply along a propagation path of, e.g., 0.5 m or less.

Furthermore, during propagation, portions of the ultrasonic signals are generally reflected at interfaces where materials of different acoustic impedances adjoin to each other. Therein, a factor of reflectance generally depends on a ratio of the acoustic impedances proximal and distal to the interface. The acoustic impedance generally relates to the product of the sound velocity and the density in a material. Both, the sound velocity and the density are significantly higher in tissue as compared to air. Accordingly, while there are generally no significant reflections of ultrasonic signals within the tissue of a patient, a very significant portion of the ultrasonic signals is reflected at an interface where tissue adjoins to air. Such interface may be, for example, an outer surface of a thorax of the patient. Alternatively such interface may occur at the lung of a patient where tissue adjoins an inner air volume of the lung. In fact, the acoustic impedances between the tissue and the air differ to such significant extend that a major portion or even almost the entire ultrasonic signals are reflected at such tissue/air interfaces.

Preferably, according to an embodiment, the ultrasonic transmitter and the ultrasonic receiver are configured such as to emit and receive, respectively, ultrasonic signals having a frequency of more than 20 kHz, preferably more than 40 kHz. Particularly, the ultrasonic signals may have a frequency of between 30 kHz and 1 Mhz, preferably between 50 kHz and 500 KHz.

On the one hand, such relatively low frequency ultrasonic signals are generally subject to relatively low attenuation upon propagation through tissue. On the other hand, a wavelength of such ultrasonic acoustic signals is generally sufficiently short such as to enable measuring short distances or measuring short-distant changes in a measured distance.

Generally, the wavelength λ correlates with a ratio of the sound velocity c and the frequency f of an acoustic wave, i.e., λ=c/f. Thus, with an assumed sound velocity in tissue of c=1500 m/s and an assumed frequency of 50 kHz, the wavelength is λ=3 cm. Accordingly, upon measuring a phase difference of ±1, distances of approximately ±0.75 cm may be measured (taking into account the to and fro motion of the signal). With an assumed frequency of 500 kHz, the wavelength is λ=3 mm and, accordingly, distances of approximately +0.75 mm may be measured.

Accordingly, using ultrasonic signals in the indicated frequency range, distances or changes in distances in the indicated order of magnitude may be measured based on analysing the phase difference between the transmitted ultrasonic signals and the received ultrasonic signals. Generally, these distances are in a same order of magnitude as distances by which, for example, a portion of the thorax or the lung of the patient moves upon respiration.

Overall, the ultrasonic signals are hardly significantly attenuated during their propagation throughout the patient's body and are mainly reflected upon reaching an outer surface of the body and/or an interface at the patient's lung. The reflected portions of the initially emitted ultrasonic signals may then be received by the ultrasonic receiver. Finally, the controller may determine the phase difference and generate the phase signal, accordingly.

Embodiments of the apnoea detection arrangement according to the second aspect of the present invention may combine the implantable apnoea detection device according to the first aspect of the present invention with an apnoea monitoring device. Such monitoring device is configured to detect the occurrence of the apnoea based on analysing the time-dependency of the phase signal generated by the controller of the apnoea detection device.

The apnoea monitoring device may be an external device. Accordingly, the apnoea monitoring device may neither have to be integrated into the implantable apnoea detection device nor have to be adapted for implantation purposes. Instead, the apnoea monitoring device may be arranged external to the body of the patient. Accordingly, the apnoea monitoring device may not have to be miniaturized. The apnoea monitoring device may communicate with the implantable apnoea detection device for exchanging signals, particularly for exchanging the phase signals. Such communication may be based, e.g., on wireless techniques.

The apnoea monitoring device may receive the phase signal from the apnoea detection device and may suitably analyse the time-dependency of this phase signal in order to detect any indicators indicating the occurrence of an apnoea.

Particularly, according to an embodiment, the apnoea monitoring device is configured to detect the occurrence of the apnoea upon the phase signal changing less than a predetermined phase change over longer than a predetermined duration.

In other words, the apnoea monitoring device may continuously or repeatedly analyse the phase signal with regard to changes occurring in this phase signal and may compare detected changes with predetermined values representing changes as they may be expected during normal respiration of the patient.

Generally, the patient continuously breathes such that his thorax is expanded and compressed periodically and his lung is inflated and deflated periodically. Accordingly, during normal respiration, the changes in the phase signal resulting from the ultrasonic signals being reflected at the moving surface of the thorax and/or at the moving interface at the lung periodically varies. The changes in the phase signal may be smaller than π, meaning that changes in a distance between the ultrasonic transmitter and receiver, on the one hand, and the thorax surface and/or the lung interface, respectively, on the other hand, are smaller than a quarter of the wavelength of the ultrasonic signal. In cases where the thorax surface and/or the lung interface moves by more than such distance during normal respiration, the changes in the phase signal may be larger than π. However, in the approach described herein, an absolute value of the changes in the phase signal is generally not of major interest. Instead, a periodicity with which the phase changes vary may have to be observed as such variation corresponds to the periodicity of the respiration of the patient.

In case, the phase signal changes by less than a predetermined phase change value, this means that an amplitude of the phase changes decreases and/or a period of the phase changes extends. In other words, reduced changes in the phase signal indicate reduced respiration motions in the patient. In an extreme case of an apnoea, the respiration suddenly stops, resulting in significantly reduced changes in the phase signal or even no changes at all in the phase signal due to the non-existing respiration motion. When such lacking respiration motion is detected for more than a predetermined duration, it may be assumed that the respiration is not only slowed down for a short period of time but, indeed, a significant apnoea occurs with the patient.

According to an embodiment, the occurrence of the apnoea is detected upon the phase signal changing less than a predetermined phase change which corresponds to the ultrasonic signal travelling along a distance of 5 mm or less, or even a distance of 2 mm or.

Expressed differently, it is assumed that an apnoea occurs when the phase signal provided by the controller of the implanted apnoea detection device changes less than a predetermined value, this value indicating that the thorax surface and/or the lung interface moves by less than a predetermined small amplitude. Such small or non-existing amplitude of the thorax or lung motion may indicate an occurrence of an apnoea.

Alternatively or additionally, the occurrence of the apnoea may be detected upon the phase signal changing less than the predetermined phase change for longer than a duration of 8 s or more or even longer than a duration of 15 s or more or longer than a duration of 30 s or more.

In other words, it is assumed that an apnoea occurs when the phase signal changes less than the predetermined value for an extended period of time of 8 s or more. In cases where the significantly reduced or non-existing respiration motion prevails for such extended period of time, this may be taken as indicating the occurrence of an apnoea.

In embodiments of the implantable blood pressure monitoring device according to the third aspect of the present invention, a blood pressure detector is combined with the implantable apnoea detection device according to an embodiment of the first aspect of the present invention. The blood pressure detector is specifically configured for measuring the blood pressure and generating a blood pressure signal indicating this blood pressure within the blood vessel and/or blood chamber of the patient's body. Accordingly, the blood pressure detector generally comprises a pressure sensor adapted for being specifically sensitive in a typical blood pressure range. The blood pressure detector may, e.g., be a MEMS. For example, the blood pressure detector may have similar or same characteristics as described in the applicant's prior application German Publication No. 10 2015 116 648 A1.

According to an embodiment, the blood pressure detector and the implantable apnoea detection device are integrated in a common housing.

In other words, the implantable blood pressure monitoring device may be an integrated device in which both, the blood pressure detector and the apnoea detection device are accommodated in the same housing and may therefore be handled as a unit. As both, the blood pressure detector and the apnoea detection device may be provided as MEMS, both components may be miniaturized such that the entire unit may be included in the housing being sufficiently small for implantation purposes. Furthermore, both components may share some structures and/or functionalities of the entire unit. For example, both components may be supplied by a common energy storage device such as a battery or capacitor.

Preferably the blood pressure monitoring device housing is made of titanium or another material suitable for implantation within a blood vessel, in particular the pulmonary artery. The pressure monitoring device may comprise a capacitive blood pressure detector (MEMS), the apnoea detection device, an electronic module and a battery. The housing may comprise (titanium) diaphragm(s) and a pressure transducer medium (in particular a fluid as silicone oil) within the housing to transfer the pressure of the blood within the blood vessel to the pressure detector. The housing may also comprise at least two expansion grooves to compensate pressure changes induced by thermal expansion of the pressure transducer medium. The blood pressure monitoring device may also comprise a fixation device to anchor the blood pressure monitoring device within the blood vessel, e.g., the pulmonary artery. The fixation device may comprise at least two fixation wires formed as loops and made of a shape memory material as Nitinol. The fixation device may be configured to change between a compressed state and an expanded state. The blood pressure monitoring device may be implanted via catheter whereby the fixation device is in the compressed state during implantation. At the implantation site the fixation device may change in the expanded state where the fixation device expands against the vessel walls to keep or anchor the blood pressure monitoring device at the implantation site.

However, in principle, it may not be excluded that the blood pressure monitoring device comprises the blood pressure detector and the apnoea detection device as separate units, which may be handled and implanted independently from each other.

In embodiments of the blood pressure monitoring arrangement according to the fourth aspect of the present invention, an embodiment of the implantable blood pressure monitoring device according to the third aspect of the present invention cooperates with a blood pressure observation device. While the blood pressure monitoring device is implanted within the patient, the blood pressure observation device generally is a separate device provided external to the patient. For example, the blood pressure observation device may be part of a health status monitoring system which may generally supervise physiological parameters serving as indicators for a current medical condition of the patient.

Therein, the blood pressure observation device is generally configured to monitor the blood pressure within the patient's blood vessel and/or blood chamber based on the blood pressure signal generated by the blood pressure detector. Accordingly, the blood pressure observation device may observe or supervise the patient's medical condition particularly with regards to abnormal conditions relating to blood pressures exceeding upper or lower limits in specific situations. Upon such monitoring of the blood pressure, the monitoring device May furthermore take into account the phase signal generated by the controller of the apnoea detection device. Accordingly, the monitoring device may determine whether or not the patient is currently subject to an apnoea. The information about the apnoea status may then be used for suitably interpreting the blood pressure signal.

For example, according to an embodiment, the blood pressure monitoring device is configured to detect an occurrence of an apnoea upon the phase signal changing less than a predetermined phase change over longer than a predetermined duration. Furthermore, the blood pressure monitoring device is configured to, upon detecting the occurrence of the apnoea, execute at least one of
marking a blood pressure signal monitored during the occurrence of the apnoea as being unreliable,
discarding a blood pressure signal monitored during the occurrence of the apnoea, and
temporarily interrupting monitoring the blood pressure during the occurrence of the apnoea.

In other words, the blood pressure monitoring device may analyse the phase signal provided by the controller of the apnoea detection device in a similar manner as described further above with regards to the apnoea detection arrangement. In case an occurrence of an apnoea is detected, the information comprised in the blood pressure signal may not be reliable as the apnoea may generally significantly influence the blood pressure. Accordingly, the current blood pressure signal may be ignored by the blood pressure monitoring device. For such purpose, the blood pressure signal may either be marked as being unreliable or may be discarded or, as a third alternative, the monitoring of the blood pressure may be temporarily interrupted. Accordingly, it may be guaranteed that no measures for influencing an excessive blood pressure are initiated in situations in which the occurrence of an apnoea temporarily influences the measured blood pressure and makes blood pressure signals unreliable.

Embodiments of the method for detecting an apnoea status of the patient according to the fifth aspect of the present invention and of the method for monitoring a blood pressure of a patient according to the sixth aspect of the present invention may be implemented, executed or controlled using embodiments of the devices and arrangements according to the first to fourth aspects of the present invention.

It shall be noted that possible features and advantages of embodiments of the present invention are described herein with respect to various embodiments of an apnoea detection device or arrangement, on the one hand, and with respect to various embodiments of a blood pressure monitoring device and arrangement, on the other hand, as well as with respect to corresponding methods. One skilled in the art will recognize that the features may be suitably transferred from one embodiment to another and features may be modified, adapted, combined and/or replaced, etc. in order to come to further embodiments of the present invention.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

In the following, advantageous embodiments of the present invention will be described with reference to the enclosed drawings. However, neither the drawings nor the description shall be interpreted as limiting the present invention.

FIG. 1 shows an apnoea detection arrangement with an implantable of apnoea detection device according to an embodiment of the present invention.

FIG. 2 shows a blood pressure monitoring arrangement with an implantable blood pressure monitoring device according to an embodiment of the present invention.

The figures are only schematic and not to scale. Same reference signs refer to same or similar features.

### DETAILED DESCRIPTION

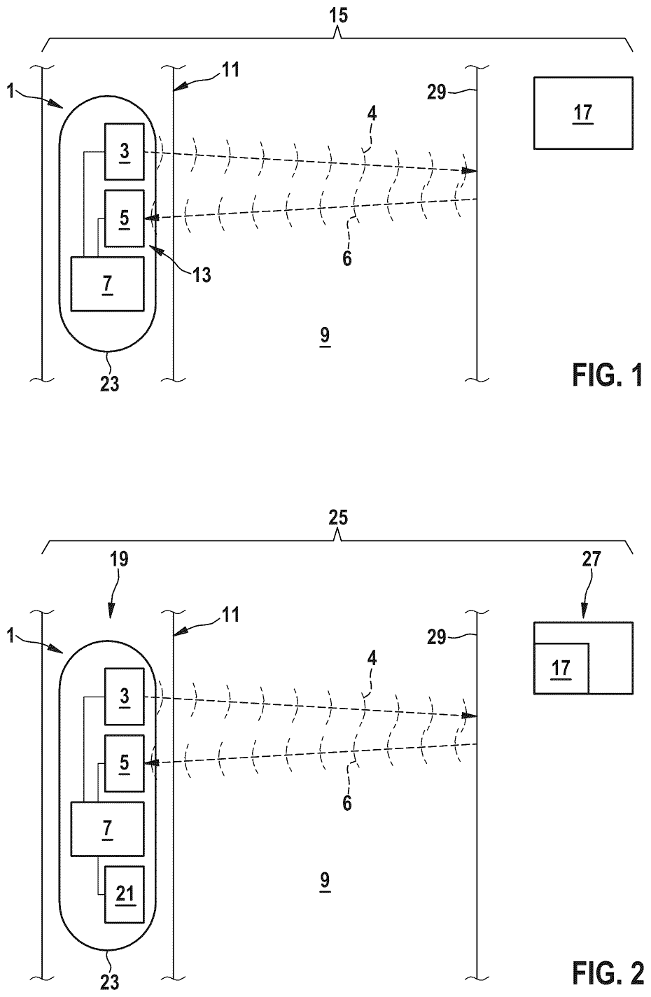

FIG. 1 shows an apnoea detection arrangement 15 including an apnoea detection device 1 and an apnoea monitoring device 17. The apnoea detection device 1 is implanted into a vessel 11 in a body 9 of a patient. The apnoea detection device 1 comprises an ultrasonic transmitter 3, an ultrasonic receiver 5 and a controller 7 comprised in a common housing 23. The ultrasonic transmitter 3 is configured for transmitting ultrasonic signals 4 towards a surface 29 of the body 9. At such surface 29, tissue within the body 9 having a high sound velocity and a high density and therefore having a high acoustic impedance adjoins to air having a low sound velocity and a low density and therefore having a low acoustic impedance. Accordingly, a major portion of the transmitted ultrasonic signals 4 is reflected at such surface 29 such that reversing ultrasonic signals 6 may be received by the ultrasonic receiver 5.

The ultrasonic transmitter 3 and/or the ultrasonic receiver 5 may be micro-electromechanical systems (MEMS). Particularly, such ultrasonic components may be provided as piezoelectric micromachined ultrasonic transducers 13 (PMUT), thereby enabling a miniaturisation of the entire implantable apnoea detection device 1 while allowing emitting ultrasonic signals with a relatively low frequency spectrum of, for example, between 20 kHz and 1 MHz.

For example, the ultrasonic signals 4, 6 may be transmitted with a frequency of f=50 KHz. For such frequency, a relatively low attenuation of between 1.5 dB and 5 dB may be expected in human tissue along a propagation path from the ultrasonic transmitter 3 to the surface 29 of the body 9 and back to the ultrasonic receiver 5. Such propagation path is typically shorter than 0.5 m and may substantially be determined by a constitution of the patient and by an implantation position. Furthermore, assuming a sound velocity of 1500 m/s, such frequency corresponds to a wavelength of 3 cm. Accordingly, a phase change of ±π corresponds to a measurable distance of ±0.75 cm.

The ultrasonic receiver 5 comprises or is connected to a phase detector. Upon receiving the reversed ultrasonic signals 6, a received signal may be supplied to the phase detector, in order to generate a signal which may be evaluated by the controller 7 and/or by the external apnoea monitoring device 17 communicating with the controller 7. Such signal is generally generated based on the detected change of the phase.

Based on such phase information, particularly based on a time-dependency of the phase signal, a respiratory motion such as a motion of the thorax or a motion of the lung may be detected. From such motion information, an information about a current respiratory activity of the patient may be derived. Upon falling below a predetermined limit value, an occurrence of an apnoea may be assumed.

FIG. 2 shows a blood pressure monitoring arrangement 25 comprising a blood pressure monitoring device 19 and a blood pressure observation device 27. The blood pressure monitoring device 19 is implanted in a vessel 11 within the body 9 of the patient. The blood pressure observation device 27 is located remotely from the patient. The blood pressure monitoring device 19 comprises a blood pressure detector 21 as well as an entire apnoea detection device 1 including the ultrasonic transmitter 3, the ultrasonic receiver 5 and the controller 7. All these components are included in an integrated unit and accommodated within a common housing 23.

The blood pressure detector 21 continuously or repeatedly generates a blood pressure signal indicating a blood pressure within the vessel 11. Furthermore, the blood pressure observation device 27 communicates with the blood pressure monitoring device 19 including the blood pressure detector 21 and the apnoea detection device 1. Accordingly, the blood pressure observation device 27 may be interpreted to include the apnoea monitoring device 17.

Overall, the blood pressure observation device 27 may monitor the blood pressure within the blood vessel 11 and may simultaneously take into account the information about the apnoea status derived from the phase signal generated by the controller 7 of the apnoea detection device 1. Upon detecting the occurrence of an apnoea, the blood pressure signal concurrently detected may be discarded or may be marked as the unreliable. As a further alternative, the monitoring of the blood pressure may be temporarily interrupted during the occurrence of the apnoea.

Finally, it should be noted that the term “comprising” does not exclude other elements or steps and the “a” or “an” does not exclude a plurality. Also elements described in association with different embodiments may be combined. It should also be noted that reference signs in the claims should not be construed as limiting the scope of the claims.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

### LIST OF REFERENCE NUMERALS

1 implantable apnoea detection device
3 ultrasonic transmitter
4 transmitted ultrasonic signals
5 ultrasonic receiver
6 reversed ultrasonic signals
7 controller
9 body of a patient
11 vessel
13 piezoelectric micromachined ultrasonic transducer
apnoea detection arrangement
17 apnoea monitoring device
19 implantable blood pressure monitoring device
15 21 blood pressure detector
23 housing
25 blood pressure monitoring arrangement
27 blood pressure observation device
29 surface of the body

1. Implantable blood pressure monitoring device configured for being implanted into a body of a patient comprising:
a blood pressure detector,
wherein the blood pressure detector is configured for generating a blood pressure signal indicating a blood pressure within one of a blood vessel and a blood chamber in the body of the patient,
and wherein the blood pressure monitoring device further comprises a apnoea detection device,
wherein the apnoea detection device comprises,
an ultrasonic transmitter,
an ultrasonic receiver, and
a controller,
wherein the ultrasonic transmitter is configured for transmitting ultrasonic signals,
wherein the ultrasonic receiver is configured for receiving ultrasonic signals, and
wherein the controller is configured for generating a phase signal indicating a phase difference between the transmitted ultrasonic signals and the received ultrasonic signals, and wherein the blood pressure detector and the implantable apnoea detection device are integrated into a common housing.

2. The implantable blood pressure monitoring device of claim 1
wherein the ultrasonic signals have a frequency of more than 20 KHz.

3. The implantable blood pressure monitoring device according to claim 1,
wherein the ultrasonic signals have a frequency of between than 30 kHz and 1 MHz.

4. The implantable blood pressure monitoring device according to claim 1 wherein at least one of the ultrasonic transmitter and the ultrasonic receiver comprises a piezoelectric micromachined ultrasonic transducer.

5. Blood pressure monitoring arrangement comprising:
an implantable blood pressure monitoring device according to claim 1, and
a blood pressure observation device,
wherein the blood pressure observation device is configured to monitor the blood pressure within one of the blood vessel and the blood chamber of the patient based on the blood pressure signal generated by the blood pressure detector and taking into account the phase signal generated by the controller (of the apnoea detection device.

6. The blood pressure monitoring arrangement of claim 5,
wherein the blood pressure observation device is configured to detect an occurrence of an apnoea upon the phase signal changing less than a predetermined phase change over longer than a predetermined duration, and
wherein the blood pressure observation device is configured to, upon detecting the occurrence of the apnoea, execute at least one of
marking a blood pressure signal monitored during the occurrence of the apnoea as being unreliable,
discarding a blood pressure signal monitored during the occurrence of the apnoea, and
temporarily interrupting monitoring the blood pressure during the occurrence of the apnoea.

7. The blood pressure monitoring arrangement of claim 5, further configured to detect the occurrence of the apnoea upon the phase signal changing less than a predetermined phase change over longer than a predetermined duration, wherein at least one of the following conditions applies:
the predetermined phase change corresponds to the ultrasonic signal travelling along a distance of 5 mm or less;
the predetermined duration is 8 s or more.

8. Method for detecting an apnoea status of a patient, the method comprising:
transmitting ultrasonic signals by an ultrasonic transmitter implanted within the patient,
receiving ultrasonic signals by an ultrasonic receiver implanted within the patient, and detecting an occurrence of the apnoea based on analyzing a time-dependency of a phase signal indicating a phase difference between the transmitted ultrasonic signals and the received ultrasonic signals.

9. Method of claim 8,
wherein the occurrence of an apnoea is detected upon the phase signal changing less than a predetermined phase change over longer than a predetermined duration.

10. Method for monitoring a blood pressure of a patient, the method
comprising:
repeatedly detecting the blood pressure within one of a blood vessel and a blood chamber of the patient by a blood pressure detector implanted within the patient,
detecting an apnoea status of the patient in accordance with claim 12,
monitoring the blood pressure based on the detected blood pressure and taking into account the detected apnoea status of the patient.

11. Method of claim 10,
wherein during monitoring the blood pressure, upon detecting the occurrence of the apnoea, at least one of the following actions is executed:
marking a blood pressure signal detected during the occurrence of the apnoea as being unreliable,
discarding a blood pressure signal detected during the occurrence of the apnoea, and
temporarily interrupting monitoring the blood pressure during the occurrence of the apnoea.
